(19) United States
(12) Patent Application Publication
(10) Pub. No.: US 2006/0127917 A1
(43) Pub. Date: Jun. 15, 2006

(54) Comprehensive virus disinfecting strategy and methods for making a preventive anti-virus medical product

(76) Inventors: Xiao-Qing Han, Naperville, IL (US); MingXia Liu, Naperville, IL (US)

Correspondence Address: Xiao-Qing Han, 1464 Larsen Court, Naperville, IL, 60563, US

(21) Appl. No.: 11/010,653

(22) Filed: Dec. 13, 2004

(51) Int. Cl. C12Q 1/70; C12Q 1/68

(52) U.S. Cl. 435/6; 435/5

(57) ABSTRACT

The present invention provides a comprehensive virus disinfecting strategy with more than two biologically active anti-virus components work synergistically to form a multi-hurdle defending line for virus disinfecting, and methods for making a medical product with anti-virus properties. The comprehensive preventive system for virus disinfecting, combines components competitively inhibiting virus attachment on cell surface, immune-boosting nutrients for strengthening immunology defense, and components defectively attacking virus infusion process. The product is an anti-influenza medical food containing from 0.01% to 3% of glycopeptide with covalently attached sialic acids and its derivatives, 0.01% to 5% of ginseng extracts, and 0.01% to 3% of polyphonies from herb extracts. The anti-influenza medical product can be administrated daily to prevent from infection with viruses.

### FIELD OF THE INVENTION

The present invention relates to a comprehensive virus disinfecting strategy and methods for making a therapeutic anti-virus product. More specifically, the present invention relates to a preventive strategy against infection with viruses by establishing a comprehensive multi-hurdle defending system with multiple biologically active anti-virus components work synergistically to combat infection with viruses, and a method for making an anti-virus medical product. More specifically, the anti-virus medical food product is a natural anti-influenza drink.

### BACKGROUND

Numerous strategies have been developed to achieve the goal of controlling virus infections, but thus far no effective antiviral agents against certain viruses have been developed, due to the fact that all viruses are highly unstable in genetic level and mutate overtime time. Influenza virus, in particular, threatens people all over the world, because it becomes prevalent on a global scale once in several years, resulting in a large number of victims. Every year, influenza epidemics are responsible for an average of approximately 20,000 deaths in the United States. Furthermore, the spread of influenza virus through a population can result in epidemics, which have considerable economic impact.

Influenza is an enveloped, single-stranded, negative-sense RNA virus. It is the only member of the Orthomyxoviridae family and has been sub-grouped into three types, A, B and C. Influenza A viruses have been isolated from many animal species in addition to humans, while the influenza B and C viruses infect mainly humans. Infection with influenza A or B often can cause a highly contagious, acute respiratory illness. Influenza virus consists of an internal ribonucleoprotein core containing a single-stranded RNA genome and an outer lipoprotein envelope lined inside by a matrix protein. The influenza virus envelope is characterized by the presence of two surface glycoproteins: hemagglutinin and neuramimidase.

The segmented genome of influenza A consists of eight molecules of linear, negative polarity, single-stranded RNA sequences that encode ten polypeptides. Among the ten segments: segment 4 of the genome consists of a 1778 nucleotide sequence encoding a 566 amino acid hemagglutin (HA) surface glycoprotein which projects from the lipoprotein envelope and mediates attachment to and entry into cells. The two glycoproteins mentioned above are necessary for the virus to enter animal cells. The surface glycoproteins of influenza A exhibit much greater variability than their homologues in the B and C viruses. Influenza A virus is further classified into subtypes depending on the antigenicities of hemagglutinin (hereinafter referred to simply as HA) and neuramimidase (hereinafter referred to simply as NA). The HA of influenza A virus comprises two structurally distinct regions, namely, a globular head region and a stem region. The globular head region contains a receptor binding site which is responsible for virus attachment to a target cell and participates in the hemagglutination activity of HA. On the other hand, the stem region contains a fusion peptide which is necessary for membrane fusion between the viral envelope and an endosomal membrane of the cell and thus relates to fusion activity (Wiley et al., Ann. Rev. Biochem., 56, 365-394, 1987).

Similarly, the segmented genome of influenza B consists of eight molecules of linear, negative polarity, single-stranded RNA sequences that encode eleven polypeptides. Influenza B viruses also contains two glycoproteins at their surface with different molecular characteristics.

The segmented genome of influenza C consists of seven molecules of linear, negative polarity, single-stranded RNA sequences that encode eight polypeptides, among that segment 4 of the genome consists of a 2074 nucleotide sequence encoding a 655 amino acid hemagglutinin-esterase surface glycoprotein that projects from the lipoprotein envelope and mediates attachment to cells, fusion, and has receptor-destroying activities.

To infect an animal cell, the envelope glycoprotein influenza hemagglutinin need to adsorbs to sialyloligosaccharide molecules in cell membrane glycoproteins and glycolipids. Specifically, influenza virus envelope protein called hemagglutinin binds to a trisaccharide structure containing sialic acids, galactose and N-acetylglucosamine, and is present as the terminal structure of large carbohydrate units of cell surface glycolipids and glycoproteins. This binding is crucial for viral infection.

Following endocytosis of the virion, a conformational change in the glycoprotein hemagglutinin molecule occurs within the cellular endosome that facilitates membrane fusion and triggers un-coating. The nucleocapsid migrates to the nucleus in which viral mRNA will be transcribed as the essential initial event in infection. Transcription and replication of influenza RNA then take place in the nucleus of infected cells and assembly into virions occurs by budding out of or through the plasma membrane. During mixed infections, viruses can re-assort genes and generate mutants.

In the United States, four antiviral agents are currently approved for preventing or treating influenza: amantadine, rimantadine, zanamivir, and oseltamivir. Amantadine and rimantadine are chemically related antiviral drugs against influenza A viruses but not influenza B viruses. After influenza A viruses enter cells, these drugs inhibit the un-coating of influenza A viruses by blocking the ion-channel activity of the viral M2 protein.

Zanamivir and oseltamivir, analogues of sialic acid, are members of a new class of antiviral agents that selectively inhibit the neuramimidase of both influenza A and B viruses. Neuramimidase cleaves terminal sialic acid residues from carbohydrate moieties on the surfaces of host cells and influenza virus envelopes, promoting the release of progeny viruses from infected cells. The proposed mechanism of action for neuramimidase inhibitors is to block the active site of neuramimidase and leave un-cleaved sialic acid residues on the surfaces of host cells and influenza viral envelopes. Consequently, viral hemagglutinin binds to the un-cleaved sialic acid residues, resulting in the formation of large viral aggregation at the host cell surface and a reduction in the amount of virus that is released and can infect other cells. By the above proposed mechanism, Zanamivir was approved for treatment of uncomplicated acute illness caused by influenza virus in persons aged greater than or equal to 12 years who have been symptomatic for no more than 2 days. And Oseltamivir was approved for treatment of uncomplicated illness caused by influenza infection in adults aged greater than or equal to 18 years who have been symptomatic for no more than 2 days.

Numerous strategies have been developed to achieve the goal of combating infection with viruses.

It has been reported that certain non-specific hemagglutination inhibition factors are contained in human milk and cow milk [Saito, et al., Agricultural Biological Chemistry, 36. 1437-1439 (1972)].

U.S. Pat. No. 5,147,853 teaches a method for protection against gastrointestinal bacteria. Epstein-Barr virus and influenza viruses that comprises administering an effective amount of an isolated and purified sialic acid-conjugated protein derived from cow milk to a patient in need thereof wherein said sialic acid-conjugated protein is a kappa-casein, a glycomacropeptide obtained by reacting rennet or pepsin with kappa-casein, cow milk, reconstituted milk or casein at pH 5.5-5.6.

U.S. Pat. No. 5,137,922 discloses a preventive and curative medicament against infection with influenza virus containing tea or tea polyphenols.

U.S. Pat. No. 5,344,820 reveals a vaccine against an infectious disease caused by virus. Since virus itself tends to undergo mutation or the like, it is not seldom that vaccination does not work well. Regarding influenza virus in particular, the type of virus which spreads changes every year and mass vaccination for schoolchildren and the like is considered to be almost meaningless.

Because genetics of viruses change over time and their surface glycoproteins exhibit great variability, any virus disinfecting line with a single target and single component always encounter challenges. Throughout recorded history influenza has remained a widespread, unpredictable disease with high morbidity and mortality, particularly among the elderly. Despite intensive screening of many thousands of substances for anti-influenza activity, no real drugs have ever been developed that can really cure infection of viruses. Currently available drugs effective in influenza treatment can only release the symptom for about one or two days, and most of them cause undesirable side effects. Therefore, problems will be encountered with the use of current drugs in general populations. The drugs need to be given very early after infection in order to be effective and need to reach the site of infection at a high enough concentration.

Currently, the main method for preventing influenza and its severe complications is influenza vaccination. While each year's influenza vaccine is based on the result of a computer model prediction on the potential of emerging influenza virus, the prediction could miss. Influenza-specific antiviral drugs are an important adjunct to vaccine but are not a substitute for vaccine. Therefore, there is a strong demand for development of an infection protectant that has no adverse effects and does not lose its preventive effects by mutation of viruses.

More importantly, there remains a need for establishing an effective strategy for virus disinfecting. Because the genetics of viruses change over time and their surface antigens exhibit great variability, any drug of combating infection with viruses using a single target and single component always has its limitations and encounters sever challenges. A more sophistic drug developing strategy need to be developed to combat the infection with viruses.

There remains a need for developing methods of screening effective anti-virus components that can work together synergistically to combat infection with viruses. There is also a need for making an effective anti-virus product. Furthermore, there remain a need for developing a preventive anti-virus medical product that can conveniently be administrated daily to prevent the infection with viruses. Additionally, there remains a need for developing anti-influenza products that can be utilized conveniently for all ages of consumer groups without any undesired side-effect.

Thus, there remains a need for developing herb-extract-based anti-influenza products that is convenient to use, save, and effective. Most importantly, there remains a need for developing a formula and methods for making a product with the advantages described above.

The current invention, through extensive investigations in the area of interface biology, the absorbance of virus particles at cell surface, immune-boosting nutrition, screening of biologically active anti-virus components, and anti-virus studies, provides a novel strategy and product concept with detailed approaches to fulfilling the above needs. The invention also provides a new concept of virus disinfecting through a routine administration of an anti-virus medical product. Finally, the invention provides methods for making an anti-influenza drink.

### SUMMARY OF THE INVENTION

The object of the present invention is to eliminate the above-mentioned problems still associated with prior arts and to provide a new strategy of virus disinfecting, and an anti-virus medical product. The present invention, as a result of extensive studies and researches, has discovered that an effective virus disinfecting strategy is to form a comprehensive hurdle system with multi-defending lines consisting of several anti-virus components work synergistically to deliver desired functions. The comprehensive hurdle system can effectively eliminates infection with viruses through a routine administration of a natural anti-virus medical product. The product compiled with multi-defending lines of anti-virus natural components is in a form that can be administrated daily to prevent infection with viruses.

According to the present invention, the strategy of combating infection with viruses is a comprehensive virus disinfecting strategy compiling more than two biologically active anti-virus components to form multi-defending hurdle lines. Based on the fact that viruses are genetically unstable, physically attached on and/or included in living cells, and can not be killed in vivo (because this will also destroy infected host cells), any conventional drug design strategy targeted on a single component and/or functional unit will not work very well as it has been confirmed by all clinical results to date. Because a drug delivered into a cell to kill infused virus could hardly be workable, the options left for an effective drug design will always try to inhibit the attachment of virus on the cell surfaces. While the mechanism of such a drug is to block the entering of viruses into cells, at the time a patient found symptom of infection with virus it will always be too late to use the drug for the purpose it to be administrated. Therefore, a better approach will always be prevention. However, a drug by its nature always means some toxicity thus should not be administrated routinely for prevention purposes.

Therefore, an ideal solution of preventing infection with viruses is to use natural ingredients, and formulated and processed in a way that can be accepted by consumers of most populations and administrated routinely without any site effects. The present invention, thus, provide a comprehensive virus disinfecting strategy and a detailed example of making an anti-virus medical product.

Although drugs of modern medicine are main resources of inhibiting the attachment of virus at cell surfaces, herbal medicines as well as other natural components have a lot to offer in treating and preventing infection with viruses. Therefore, the invention also includes the screening of anti-virus herb medicines to be formulated into an orally administrable anti-virus product. The invention formula also includes additives for keeping the product stable in its shelf-life, preventing oxidation of biologically active ingredients, and stabilizing the product in united liquid form. The invention includes stabilized liquid products with improved storage characteristics that are rheologically stable. The invention also includes methods for incorporating the extract of anti-virus herb extracts into the product. Finally, the invention includes methods for making the product.

In one aspect, the present invention provides a comprehensive virus disinfecting strategy. The new strategy targets on more than two drug targets simultaneously to form a multiple-hurdles defending system. The multiple-hurdles defending system compiles more than two biologically active anti-virus components. The biologically active anti-virus components included in the multi-hurdle system are able to work synergistically to form a much stronger defending system against infection with virus than that of any conventional drugs. The biologically active anti-virus components included in the comprehensive multi-hurdle system are stable during production process and storage of the product. More importantly, the biologically active anti-virus components are able to work synergistically without any conflictions in order to maximize their biological functions.

In one embodiment of this aspect of the invention, the product ingredients subject to form multi-hurdle defending lines against virus infection are all food grade ingredients and the product is a functional or medical product. In another embodiment, the biologically active ingredients subject to form multi-hurdle defending lines against virus infection are medical reagent components and the product is a wide-spectra anti-virus medical product.

In another aspect, the present invention is an anti-influenza product capable of disinfecting influenza viruses in vivo and the anti-influenza product is in a liquid form that can be administrated daily to combat infection with influenza viruses.

### BRIEF DESCRIPTION OF THE DRAWINGS

FIG. 1 is a flow diagram of one embodiment of a method for making the anti-influenza product.

### DESCRIPTION OF THE PREFERRED EMBODIMENTS

Comprehensive prevention strategy. In one aspect, the current invention provides a new strategy of combating infection with viruses compiling more than two biologically active anti-virus components in a system to form a comprehensive multi-hurdle defending line. In one embodiment of this aspect of the invention is directed to a method for designing anti-virus drug, wherein the method is a comprehensive prevention strategy. In one embodiment, the biologically active anti-virus components included in the system are able to work synergistically to form multiple defending lines against infection with viruses, wherein the anti-virus components are stable during production process and during storage of the product. In another embodiment of this aspect of the invention is a new strategy of virus disinfecting, wherein the disinfecting line composes more than two anti-virus biologically active components work synergistically to prevent infection with viruses. While the strategy of all anti-virus drugs currently on market is mostly single-targeted drug at a specific binding site, the comprehensive prevention strategy emphases on a “team work” of more than two medically active components targeted at more than two binding sites under different principles.

In another aspect, the comprehensive virus disinfecting strategy is an in-vivo hurdle theory of combating infection with viruses, wherein each biologically active anti-virus component forms a hurdle to prevent from the “passing through” of viruses, and they together form a comprehensive multi-hurdle defending line against infection with viruses. In one embodiment of this aspect of the invention is directed to a strategy of forming a comprehensive multi-hurdle defending to combat infection with viruses, wherein all the hurdles work synergistically to form a virus disinfecting line in vivo. In another embodiment of the invention, the comprehensive virus disinfecting strategy is a multi-hurdle system formed by more than two disinfecting components.

In another aspect, the current invention provides a method for preventing infection with influenza viruses in a subject; said method comprises compiling more than two biologically active virus disinfecting ingredients in a system. In one embodiment of this aspect of the invention directed to a method for preventing infection with viruses, the subject is a mammal, including a human. In another embodiment of this aspect of the current invention, the viruses are all kinds of viruses capable of infecting a mammal, including a human influenza virus, and the viruses including influenza virus A. B. and C.

Virus disinfecting strategy. In one aspect, the current invention is a strategy for combating infection with viruses through a preventing approach. Because viruses are genetically unstable and can not be killed once their genetic information being integrated with host cells, conventional virus disinfecting drugs can only improve the recovery of infected patients. Because the mechanism of conventional drugs is to block the entering of virus into cells, at the time when a patient found symptom of virus infection it will be too late to use the drug for the purpose it to be administrated. Therefore, a better strategy for virus disinfecting will be prevention. In one embodiment of this aspect of the invention, therefore, is a new strategy of virus disinfecting directed to a method of creating a natural product with the capability of virus disinfecting. In one embodiment, the virus disinfecting strategy is a strategy of prevention and the prevention strategy is a comprehensive multi-hurdle strategy as disclosed herein. In another embodiment of this aspect of the invention is a combination of different virus disinfecting components working under different mechanisms to combat infection with viruses including a kinetic inhibition of virus attachment on cell surface through a competitive inhibition.

In another aspect, the current invention provides a method for preventing the infection of influenza viruses in a subject, said method comprises compiling more than two biologically active virus disinfecting natural ingredients in a system. In one embodiment of this aspect of the invention directed to a method for preventing infection with viruses, the subject is a mammal, including a human. In another embodiment of this aspect of the current invention, the viruses are all kinds of viruses capable of infecting a mammal, including a human. influenza viruses, and the viruses including influenza viruses. In another embodiment of this aspect of the invention is a combination of all natural virus disinfecting components that can be administrated routinely without any toxicity and undesired side-effects. The routinely administrated formulated product therefore will work as a safeguard in vivo to eliminate infection with viruses. It is very important that the current invention provides a solution to combat infection with viruses through a non-drug approach, from which it won't have any undesired side-effect related to a drug. Through an extensive research and screening of extracts from natural sources, this invention has provided a non-drug natural product for combating infection with viruses.

Anti-influenza medical products. In one aspect, the current invention provides a method for treating a condition associated with infection with viruses in a subject, said method comprises administering to the subject an amount of therapeutic compound as described herein. In one embodiment of this aspect of the invention provides a method for preventing infection with influenza viruses, the subject is a mammal, including a human. In another embodiment of this aspect of the invention directed to a method for preventing infection with viruses, including human influenza viruses. In another embodiment of this aspect of the current invention, the condition associated with influenza virus including a human influenza A, B, and C.

In one aspect, the current invention is a therapeutic anti-virus medical product for treating a condition associated with virus infection in a subject the subject is a mammal, including a human. In one embodiment of this aspect of the invention, the product is a therapeutic medical product with the capabilities of preventing and treating a condition associated with infection with viruses in a subject including a human. In another embodiment of this aspect of the current invention, the product is a therapeutic medical product in a form such as liquid, powder, tablet, and capsule. In another embodiment of this aspect of the current invention, the product is a therapeutic medical food product in a liquid form.

In another aspect, the current invention provides a method for making a medical product for virus disinfecting in a subject, said product comprises more than two therapeutic compound as described herein. In one embodiment of this aspect of the invention, the product contains biologically active virus disinfecting compounds including herb extracts and a glyco-peptide from milk, said herb extracts contain virus disinfecting therapeutic compound as described herein, and glyco-peptide is a peptide from the hydrolysis of a milk protein. The herb extract described herein include extracts of pin corn (Pinus parviflora), Ginseng roots, Kola nut, Sage, Ginger roots, white Willow, tea, Curcumin, and Boswellia separately or in combinations. In another embodiment of this aspect of the current invention, the therapeutic herb extract compounds are formulated together with the glyco-peptide as described herein. The glyco-peptide is a hydrolyzed peptide from milk and covalently attached with sialic acids.

In another aspect, the current invention provides a formula for treating a condition associated with infection with viruses in a subject, said the formula includes additives for keeping the product stable during its shelf-life, preventing oxidation of biologically active ingredients, and stabilizing the product in united liquid form. In one embodiment of this aspect of the invention is a method for making an anti-influenza medical product and the product is an anti-influenza drink with influenza prevention properties when it is administrated routinely during an influenza season. In another embodiment of this aspect of the current invention, the anti-influenza drink is medical product for treating infections of influenza viruses including a human influenza A, B, and C. While the product is a soft-drink product that can be consumed daily, infection with viruses can be prevented by this administration. Because human influenza viruses periodically changes types of their antigens (surface glycoproteins) and thus causes wide prevalence. It is often observed that vaccinization before winter may produces no effect, since the prevalence is caused by a virus of a different type. Therefore, a preventive strategy of virus disinfecting applying a medical food product has several advantages over any conventional virus disinfecting drug approaches discovered to date. One important embodiment of the invention, therefore, is a medical natural product and also a principal feature of the infection protectant of the present invention that a glycopeptide derived from milk can work synergistically with herb extracts in a virus disinfecting system to form comprehensive multi-hurdle virus disinfecting line against virus infection.

The following examples describe and illustrate the methods and compositions of the invention. These examples are intended to be merely illustrative of the present invention, and not limiting thereof in either scope or spirit. Unless indicated otherwise, all percentages are by weight. Those skilled in the art will readily understand that variations of the materials, conditions, and processes described in these examples can be used.

### EXAMPLE 1

### Making of an Anti-Influenza Concentrate

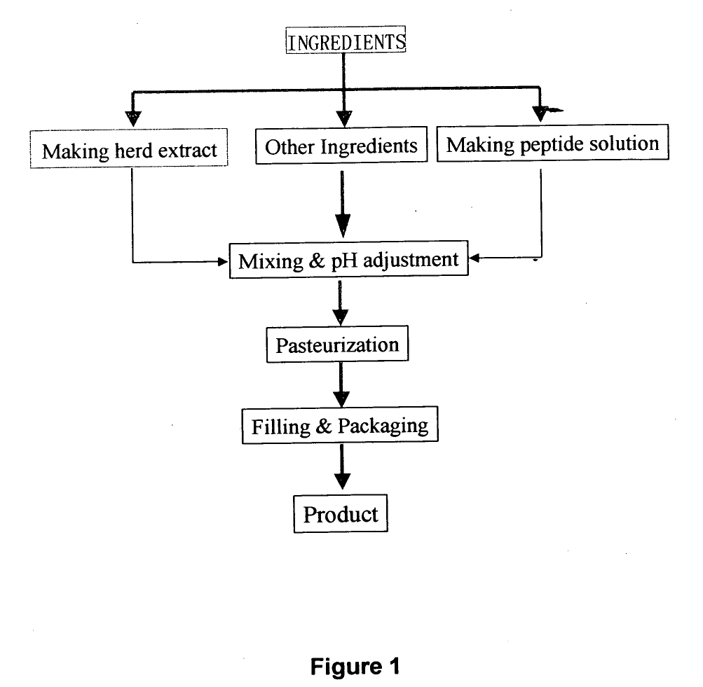

The process for making an anti-influenza concentrate is illustrated in FIG. 1, which include the following procedures:

1) Making herb extracts. Herbs such as pin corn (Pinus parviflora), Ginseng roots, Kola nut, Sage, Ginger roots, white Willow, tea, Curcumin, and Boswellia separately or in combinations are extracted by hot water and the extract condensed to certain degree. The extract, if use immediately, dose not need to be condensed and can mix directly with other ingredients in the formula. In this specific example, hers of pin corn (Pinus parviflora), Ginseng roots, Kola nut, and Sage were extracted separately in hot water (90° C.) for 30 min. The ratio of herb to water is in the range of 1:6. Mix 200 ml of each of the extracts herbs as Solution A.
2) Making glycopeptide solutions. A commercially available glycopeptide was used for the test. The glycopeptide (SA-1000) is a product from Interface Protein Technology, Inc. (Ningbo City, Zhejiang Province, P.R. China). A 6.0 grams of SA-100 was mixed with 190 ml water to obtain a mixture as make Solution B.
3) Adjusting sweetness and acidity. Mix Solution A and B, add 60 grams of sugar to obtain sweet flavor for the product. The pH of the liquid product was adjusted by citric acid to 4.8.
4) Pasteurization. The above formulated liquid was heated to boiling for 10 seconds, filled to glass containers and cool down to room temperature. The Pasteurized product was stored in refrigerator for further tests and studies.

### EXAMPLE 2

Challenge studies of an anti-influenza concentrate on MDCK cells with human influenza A.
1) Host Cells: MDCK, canine kidney. The host MDCK cells were pretreated with reagent for 0.5 hour at 2× concentration, before the virus was added.
2) Virus: Influenza Type A, strain PR8.
3) The reagents were tested treatment at dilutions of 1/5, 1/10, 1/20, and 1/40.
4) Virus inoculums without experimental compound were used as positive control.
5) Cyto-toxicity was determined by microscopic inspection.
6) Cells-only controls with no reagent or virus were used as negative controls.
7) Culture supernatants were used to run hemagglutinin assay (HA).
8) The experimental reagents (anti-influenza drink concentrate) were present during the time of viral absorption and in the media for the duration of the assay.
   Results of the Tests:

Results of the challenge study are summarized in Table 1.

TABLE 1
Antiviral and cytotoxicity results on day 7.

| | | Aggluti- | | Aggluti- |
|---|---|---|---|---|
| Reagent | | nation/ | Cytotoxicity* | nation/ |
| dilutions | CPE*/virus | Virus | no virus | no virus |
| 1/5 | Not readable** | No | Not readable | No |
| 1/10 | Cytotoxicity | No | Yes | No |
| 1/20 | None | No | None | No |
| 1/40 | None | No | None | No |
| No reagent | Yes | Yes | None | No |
| CPE—cytopathological effect. | | | | |
| *CPE cannot be evaluated in the presence of cytotoxicity. | | | | |
| **Not readable due to density of reagent. | | | | |
| Bold type indicates where reagent was effective against the virus. The results indicate that the tested reagent at 1/20 and 1/40 dilutions were not cytotoxic and showed antiviral activity against Influenza A. | | | | |
| # No antiviral activity were detected when the active components in the reagent was tested separately with the designed challenge study model (data not presented). | | | | |

What is claimed is:

1. A comprehensive virus disinfecting strategy to prevent infection with viruses, wherein principle of the strategy is the design of a multi-hurdle defending system compiling more than two biologically active anti-virus components working synergistically to eliminate infection with viruses, wherein the multi-hurdle defending line includes:
A) Components inhibiting virus attachment on cell surface;
B) Immune-boosting nutrients for strengthening immunology defense, and;
C) Components defectively attacking virus infusion process.

2. An herb extract based anti-virus medical product for treating a condition associated with infection of viruses in a subject, wherein the product containing:
A) 0.01% to 3% of glycopeptide with covalently attached sialic acids and their derivatives;
B) 0.01% to 5% of ginseng extracts; and,
C) 0.01% to 3% of polyphonies from herb extracts.

3. A method of virus disinfecting as claimed in claim 1, wherein the strategy is a comprehensive preventive strategy with more than two biologically active anti-virus components in the system working synergistically against infections of viruses.

4. A method of virus disinfecting as claimed in claim 1, wherein the biologically active anti-virus components in the system aim at different targets and work under different mechanisms in terms of their anti-influenza activities to form a multi-hurdle system against infection with viruses.

5. A method of virus disinfecting as claimed in claim 1, wherein the mechanism of virus disinfecting includes competitive inhibitions of virus attachment on cell surface, wherein the inhibitors include glycopeptides with covalently attached sialic acids and their derivatives.

6. A method of virus disinfecting as claimed in claim 1, wherein the mechanism of virus disinfecting includes immune-boosting nutrients for strengthening immunology defense, wherein the immune-boosting nutrients include water soluble extracts of ginseng roots.

7. A method of virus disinfecting as claimed in claim 1, wherein the mechanism of virus disinfecting includes components defectively attacking virus infusion process, wherein the components include polyphenols and their derivatives.

8. An herb extract based anti-virus medical product as claimed in claim 2, wherein the medical product is a therapeutic anti-virus product for treating a condition associated with infection with viruses in a subject, the subject is a mammal, including a human.

9. An herb extract based therapeutic anti-virus product as claimed in claim 2, wherein the medical product is for treating a condition associated with infection with viruses, wherein the viruses are influenza viruses including a human influenza A, B, and C.

10. An herb extract based therapeutic anti-virus product as claimed in claim 2, wherein the medical product is in a liquid form, a tablet, a powder, or a capsule, wherein the anti-virus medical product contains 0.01% to 3% of glycopeptide with covalently attached sialic acids and their derivatives, 0.01% to 5% of ginseng extracts, and 0.01% to 3% of polyphonies from herb extracts.

11. An herb extract based therapeutic anti-virus product as claimed in claim 2, wherein the product is a drink in a liquid form, wherein the anti-virus medical drink contains 0.01% to 2% of glycopeptide with covalently attached sialic acids and their derivatives, 0.01% to 3% of ginseng extracts, and 0.01% to 1% of polyphonies from herb extracts.

12. An herb extract based therapeutic anti-virus product as claimed in claim 11, wherein the product is a drink in a liquid form with pH in the range from 3.8 to 6.8,

13. An herb extract based therapeutic anti-virus product as claimed in claim 11, wherein the product is a drink in a liquid form packed in a container that can be retorted or heat filled.

14. An herb extract based therapeutic anti-virus product as claimed in claim 2, wherein the product is a drink in a powder form, wherein the anti-virus medical product contains 0.1% to 3% of glycopeptide with covalently attached sialic acids and their derivatives, 0.1% to 3% of ginseng extracts, and 0.1% to 3% of polyphonies from herb extracts.

15. An herb extract based therapeutic anti-virus product as claimed in claim 2, wherein the product is a drink in a powder form, wherein the anti-virus medical product contains 0.1% to 3% of glycopeptide with covalently attached sialic acids and their derivatives, 0.1% to 3% of ginseng extracts, and 0.1% to 3% of polyphonies from herb extracts.

16. An herb extract based therapeutic anti-virus product as claimed in claim 2, wherein the product is a tablet, wherein the tablet anti-virus medical product contains 0.1% to 3% of glycopeptide with covalently attached sialic acids and their derivatives, 0.1% to 3% of ginseng extracts, and 0.1% to 3% of polyphonies from herb extracts.

17. An herb extract based therapeutic anti-virus product as claimed in claim 2, wherein the product is a capsule, wherein the anti-virus capsule product contains 0.1% to 3% of glycopeptide with covalently attached sialic acids and their derivatives, 0.1% to 3% of ginseng extracts, and 0.1% to 3% of polyphonies from herb extracts.

18. An herb extract based therapeutic anti-virus product as claimed in claim 2, wherein the product can be administrated daily to prevent infection with viruses including human influenza viruses.
